(19) United States
(12) Patent Application Publication
(10) Pub. No.: US 2025/0269049 A1
(43) Pub. Date: Aug. 28, 2025

(54) CD20-TARGETED ANTIBODY COUPLING PHARMACEUTICAL PREPARATION

(71) Applicant: ZHEJIANG TERUISI PHARMACEUTICAL INC., Zhejiang (CN)

(72) Inventors: Kai HUANG, Zhejiang (CN); Lang ZHOU, Zhejiang (CN); Yun TANG, Zhejiang (CN); Hui ZHAO, Zhejiang (CN); Shicheng LIU, Zhejiang (CN); Jingyao HUANG, Zhejiang (CN); Yujie ZHANG, Zhejiang (CN)

(73) Assignee: ZHEJIANG TERUISI PHARMACEUTICAL INC., Zhejiang (CN)

(21) Appl. No.: 19/203,229

(22) Filed: May 9, 2025

Related U.S. Application Data
(63) Continuation-in-part of application No. 16/486,862, filed on Feb. 25, 2020, now Pat. No. 12,324,840.

(30) Foreign Application Priority Data
Feb. 20, 2017 (CN) 201710088853.9

(51) Int. Cl. A61K 47/68; A61P 35/00; C07K 16/28

(52) U.S. Cl. CPC A61K 47/68031; A61K 47/6849; A61K 47/6889; A61P 35/00; C07K 16/2887

(57) ABSTRACT

Provided is a CD20-targeted antibody-drug conjugate and a pharmaceutical composition comprising thereof, as well a method for preparing the same. The antibody-drug conjugate of the present invention has a specific DAR component distribution optimized for drug loading, and is produced by accurately controlling the feed ratio. The antibody-drug conjugate of the present invention has excellent drug activity, good stability, low toxicity and good drug development properties.

### CROSS-REFERENCE TO RELATED APPLICATION

This is a continuation-in-part application of U.S. patent application Ser. No. 16/486,862 filed on Feb. 25, 2020. The prior application Ser. No. 16/486,862 is a 371 application of International Application No. PCT/CN2018/076835, filed on Feb. 14, 2018, which claims the priority benefits of China Application No. 201710088853.9, filed on Feb. 20, 2017. The entirety of each of the above-mentioned patent applications is hereby incorporated by reference herein and made a part of this specification.

### FIELD OF THE INVENTION

The present invention relates to the field of bio-medicine and in particular to a CD20-targeted antibody coupling pharmaceutical preparation.

### BACKGROUND OF THE INVENTION

Lymphomas are a group of malignant tumors that originate from lymph nodes and other lymphoid tissues outside the nodes, with a large variety and high incidence. Every year, tens of thousands of people in China lose their lives due to lymphomas. CD20 is a non-glycosylated quadruple transmembrane phosphoprotein that is specifically expressed on the surface of B lymphocytes, and has important regulatory effects on the differentiation and proliferation of B lymphocytes. The stable and specific expression of CD20 on the surface of B cells makes it an ideal target for the treatment of B cell lymphoma. At present, Rituximab, Zevalin, Bexxar and other anti-CD20 monoclonal antibodies approved by the FDA for the treatment of B-cell lymphoma are marketed.

Antibody-drug conjugate (ADC) belongs to a new type of anticancer drug developed in recent years. It connects antibodies and cytotoxic drugs through conjugates. The targeting effect of antibodies will target cytotoxic drugs to tumor sites. ADC drugs release toxins after entering tumor cells through endocytosis and kill target cells, thereby reducing non-specific systemic toxicity common in drugs used in chemotherapy.

Therefore, those skilled in the art are committed to developing new and more effective antibody-drug conjugates targeting CD20.

### SUMMARY OF THE INVENTION

An object of the present invention is to provide an anti-CD20 antibody-drug conjugate with a specific DAR component distribution optimized for drug loading, which has excellent drug activity, good stability, low toxicity, and good drug-developing properties.

A nother object of the present invention is to provide an antibody-drug conjugate based on sulfhydryl group conjugating technology and a pharmaceutical product thereof, which produces an antibody-drug conjugate with a specific DAR component distribution accurately controlled by controlling the feed ratio.

In the first aspect of the invention, it provides an antibody-drug conjugate with a structure as shown in formula I, or pharmaceutically acceptable salt thereof:

mAb−(L−D)n I

wherein,

mAb represents an anti-CD20 antibody;

D represents a small molecule toxin;

L is a linker connecting the anti-CD20 antibody and the small molecule toxin, wherein the linker is selected from maleimidocaproyl-valine-citrulline-p-aminobenzyloxycarbonyl;

n is an average number of the small molecule toxins conjugated to the antibody;

“-” is a bond

wherein an average ratio of small molecule toxin to antibody (DAR) of the antibody-drug conjugate is in a range of 4.2±1, preferably, 4.2±0.5.

In an embodiment of the present invention, the antibody-drug conjugate comprises a mixture of components with different DAR values: DAR0, DAR1, DAR2, DAR3, DAR4, DAR6, DAR8, and wherein main DAR components in the antibody-drug conjugate has a percentage range of: 14-28% for component of DAR 2, 34-44% for component of DAR 4, 17-26% for component of DAR 6, and 9-15% for component of DAR 8, based on DAR values calculated from peak areas of hydrophobic interaction chromatography (HIC) for the antibody-drug conjugate or pharmaceutically acceptable salt thereof.

In an embodiment of the present invention, the anti-CD20 antibody in the above antibody-drug conjugate comprises a recombinant antibody, a monoclonal antibody, a humanized antibody, preferably, the anti-CD20 I antibody may be rituximab or a biosimilar thereof.

In an embodiment of the present invention, the above antibody-drug conjugate may have an average DAR value of 4.2±0.5, preferably, 4.2±0.3, more preferably, preferably, 4.2±0.2, or 4.2±0.1.

In another embodiment of the present invention, the above antibody-drug conjugate may have an average DAR value of 3.98-4.66, or any of value therebetween, for example, 4.0, 4.1, 4.2, 4.3, 4.4, 4.5 or 4.6, preferably, 4.2.

In another embodiment of the present invention, the above antibody-drug conjugate may have a structure shown as the following formula:

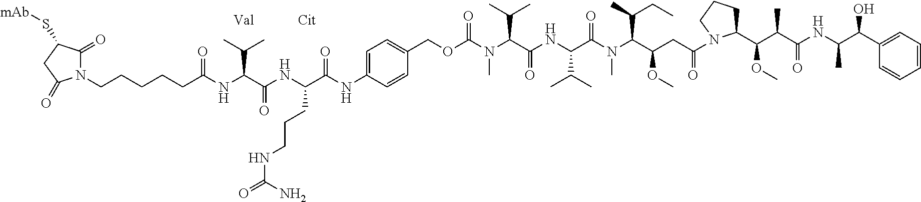

wherein mAb may be rituximab or a biosimilar thereof, wherein the average DAR value is 3.98-4.66.

In further embodiment of the present invention, the DAR components in the above antibody-drug conjugate may have a percentage range of: 1.6-5.50% for component of DAR 0, 0.2-1.3% for component of DAR 1, 16-26.5% for component of DAR 2, 1.8-5.2% for component of DAR 3, 36-43.5% for component of DAR 4, 18-25.5% for component of DAR 6, and 9-15% for component of DAR 8, based on DAR values calculated from the peak areas of hydrophobic interaction chromatography (HIC) for the antibody-drug conjugate.

In another embodiment of the present invention, the small molecule toxin in the above antibody-drug conjugate comprises monomethylauristatin, preferably, the small molecule toxin is selected from monomethylauristatin-E (MMAE), monomethylauristatin-D (MMAD), monomethylauristatin-F (MMAF), or a combination thereof. Preferably, the small molecule toxin may be monomethyl auristatin-E (MMAE).

In an embodiment of the present invention, component of DAR 4 in the above antibody-drug conjugate comprises 35-43% of the antibody-drug conjugate, and/or wherein a naked antibody without conjugation with MMAE comprises less than 3% of the antibody-drug conjugate.

In the second aspect of the invention, it provides an antibody-drug conjugate targeting CD20 or pharmaceutically acceptable salt thereof, wherein the antibody-drug conjugate has a structure as shown in formula I:
mAb−(L−D)n (I)

wherein,

mAb represents a recombinant anti-CD20 monoclonal antibody, which is rituximab or a biosimilar thereof;

D represents a small molecule toxin, which is monomethyl Auristatin-E (MMAE);

L is maleimidohexanoyl-valyl-citrulline-p-aminobenzyloxycarbonyl;

the above antibody-drug conjugate comprises or consists of a mixture of the antibody-drug conjugates targeting CD20 with diverse DAR values;

n is the average number of the small molecule toxins conjugated to the antibody;

the antibody-drug conjugate has an average DAR value of 4.2±0.5; and

“-” is a bond.

In a preferred embodiment, the antibody-drug conjugate compound targeting CD20 has an average DAR value of 3.98-4.66.

In another preferred embodiment, the structure of the antibody-drug conjugate is shown as the following formula:

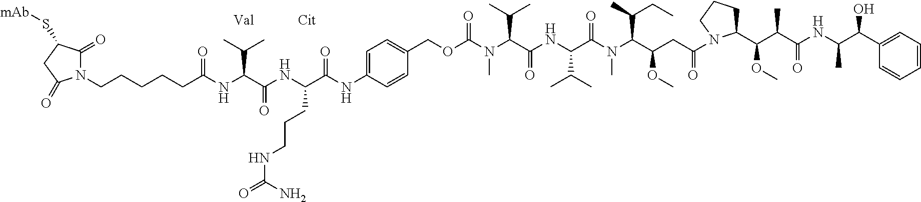

wherein, mAb is rituximab or a biosimilar thereof, and/or wherein the average DAR value is 3.98-4.66, and/or a component with 4 MMAE in the antibody-drug conjugate has the highest (about 35.54-43.04%) percentage in the conjugate mixture, and/or a naked antibody not conjugated with MMAE comprises less than about 3% of the antibody-drug conjugate mixture.

In another preferred embodiment, the antibody-drug conjugate comprises a mixture of components with different DAR values, and the DAR values are calculated from the peak areas of hydrophobic interaction chromatography (HIC), based on which, the percentage range of the component of each DAR is determined: 1.5-5.5% for component of DAR 0, 0.2-1.5% for DAR 1, 15-26.5% for DAR 2, 1.8-5.20% for DAR 3, 35-43% for DAR 4, 18-25.5% for DAR 6, and 9-15% for DAR 8, preferably, 1.66-5.50% for component of DAR 0, 0.23-1.32% for DAR 1,15.78-26.11% for DAR 2, 1.88-5.19% for DAR 3, 35.54-43.04% for DAR 4, 18.87-25.36% for DAR 6, and 9.08-14.84% for DAR 8.

In the third aspect of the present invention, it provides a method for the preparation of an antibody-drug conjugate targeting CD20 or a pharmaceutically acceptable salt thereof, wherein, method includes the following steps:
(1) reduction of a recombinant anti-CD20 monoclonal antibody and a reducing agent in a PB reaction buffer, the reducing agent was tris(2-carboxyethyl)phosphine (TCEP), and a molar ratio of the recombinant anti-CD20 monoclonal antibody to the reducing agent was 1:2.9 to 1:3.1;
(2) adding a mixture of vcMMAE and solvent dropwise to the reaction system of step (1) so that the reduced recombinant anti-CD20 monoclonal antibody and vcMMAE undergo a conjugation reaction; wherein the molar ratio of recombinant anti-CD20 monoclonal antibody to vcMMAE was 1:7.0 to 1:8.0;
(3) terminating the conjugation reaction by adding a termination buffer to the reaction system of step (2);
(4) after termination of the conjugation reaction in step (3), performing buffer exchange, transferring the conjugate solution to a TFF system, changing the PB reaction buffer to a preparation buffer and then filtering.

In another preferred embodiment, the molar ratio of recombinant anti-CD20 monoclonal antibody to tris(2-carboxyethyl)phosphine (TCEP) was 1:3; the molar ratio of recombinant anti-CD20 monoclonal antibody to vcMMAE was 1:7.5.

In another preferred embodiment, in step (1), the recombinant anti-CD20 monoclonal antibody and the reducing agent were subjected to a reduction reaction in a PB reaction buffer at pH 7.6 for 90 min at 25° C., and the molar ratio of recombinant anti-CD20 monoclonal antibody to tris(2-carboxyethyl)phosphine (TCEP) was 1:3; in step (2), the reduced recombinant anti-CD20 monoclonal antibody and vcMMAE were subjected to a conjugation reaction at 4° C. for 60 min, and the molar ratio of recombinant anti-CD20 monoclonal antibody to vcMMAE was 1:7.5.

In another preferred embodiment, the method includes the following steps:
(1) the recombinant anti-CD20 monoclonal antibody and the reducing agent were subjected to a reduction reaction in a PB reaction buffer at pH 7.6 for 90±10 minutes at 25±1° C. to obtain a reaction system containing the reduced recombinant anti-CD20 monoclonal antibody; the recombinant anti-CD20 monoclonal antibody was rituximab or its biosimilars, the reducing agent was tris(2-carboxyethyl)phosphine, and the molar ratio of the recombinant anti-CD20 monoclonal antibody to the reducing agent was 1:2.9 to 1:3.1;
(2) a solution of vcMMAE in acetonitrile and water was added dropwise to the reaction system of step (1) so that the reduced recombinant anti-CD20 monoclonal antibody and vcMMAE were subjected to a conjugation reaction for 60±10 minutes at 4±0.5° C.; the volume ratio of acetonitrile to water was 1:1, and the molar ratio of recombinant anti-CD20 monoclonal antibody to vcMMAE was from 1:7.0 to 1:8.0;
(3) L-cysteine termination buffer was added to the reaction system of step (2) to terminate the conjugation reaction, and the L-cysteine termination buffer was prepared by dissolving L-cysteine in a DTPA solution; and
(4) after termination of the conjugation reaction in step (3), buffer exchange was performed by transferring the conjugate solution to a TFF system, changing the PB reaction buffer to a preparation buffer, and then filtering.

In another preferred embodiment, the preparation buffer was HT formulation buffer, 1 L HT preparation buffer contains L-histidine 3.18 g, alginate dihydrate 70 g and Tween 80 0.2 mL, pH was adjusted to 6.5 using 0.5 mol/L hydrochloric acid and the HT preration buffer filtered through 0.22 μm membrane.

In another preferred embodiment, wherein, the filtrating in step (4) was done using a 0.22 micron Rapid-Flow unit filtration or other commonly used means in the art.

In the fourth aspect of the present invention, it provides a pharmaceutical composition, the pharmaceutical composition comprises an antibody-drug conjugate targeting CD20 or a pharmaceutically acceptable salt thereof of the present invention as above described, and a pharmaceutically acceptable carrier, a diluent, a stabilizer or an excipient. Preferably, the pharmaceutically acceptable carrier comprises water, a buffer.

In an embodiment of the present invention, the pharmaceutical composition further comprises histidine hydrochloride, alginate and tween 80.

In the fifth aspect of the present invention, it provides use of an antibody-conjugated drug targeting CD20 or a pharmaceutically acceptable salt or pharmaceutical composition comprising the same in preparation of an antitumor drug for treating a tumor in a subject.

In the sixth aspect of the present invention, it provides a method for treating a tumor in a subject in need thereof, comprising administrating an antibody-drug conjugate or a pharmaceutically acceptable salt thereof or a pharmaceutical composition of the present invention as described above to the subject.

In an embodiment of the present invention, the tumor comprises a CD20 positive tumor lymphoma or leukemia.In a preferred embodiment, the tumor is a CD20 positive tumor, lymphoma or leukemia including but not limited to B-cell lymphoma, non-Hodgkin's lymphoma or chronic lymphocytic leukemia.

In a more preferred embodiment, the tumor is B-cell non-Hodgkin's lymphoma or chronic lymphocytic leukemia.

In an embodiment of the present invention, the method comprises administrating the antibody-drug conjugate or the pharmaceutical composition to the subject via parenteral route.

In another embodiment of the present invention, the method comprises administrating the antibody-drug conjugate or the pharmaceutical composition to the subject via intramuscular, intraperitoneal, intravenous, subcutaneous, intradermal, local route.

In another embodiment of the present invention, the method comprises administrating the antibody-drug conjugate or the pharmaceutical composition in an effective dose of 0.001-3 mg/kg of body weight.

### DESCRIPTION OF THE FIGURES

FIG. 1 shows drug to antibody ratios of TRS005 antibody-drug conjugate measured by hydrophobic interaction chromatography.

FIG. 2A shows a scatter plot of flow cytometry;

FIG. 2B shows a single-parameter histogram;

FIG. 2C shows internalization efficacy of TRS005 antibody-drug conjugate of the present invention into Ramos cells measured by flow cytometer;

FIG. 2D shows internalization efficacy of TRS005 antibody-drug conjugate of the present invention into Raji cells measured by flow cytometer;

FIG. 3 shows a tumor volume-time curve (6 injections over 21 days of treatment); animal model: Daudi graft tumor in BALB/c nude mice;

FIG. 4 shows a scatter plot of tumor weight (6 injections over 21 days of treatment); animal model: Daudi graft tumor in BALB/c nude mice;

FIG. 5 shows possible toxin binding sites and the number of drugs carried by the formed monoclonal Antibody-drug conjugates.

### DETAILED DESCRIPTION OF THE INVENTION

The inventor has unexpectedly discovered through extensive and in-depth research an efficient antibody-drug conjugate (ADC) targeting CD20 with optimized toxin loading capacity. Based on this discovery, the present invention has been completed.

### Antibodies

The antibody suitable for use in the present invention is an antibody targeting CD20, a recombinant anti-CD20 monoclonal antibody. The recombinant anti-CD20 monoclonal antibody can be rituximab or its biosimilar.

### Rituximab

Rituximab of the present invention refers to a monoclonal antibody drug targeting CD20 developed by Roche and approved for marketing by the drug regulatory authority under the brand name “Rituxan”, and only the branded drug can be called rituximab.

### Rituximab Biosimilars

As used herein, unless otherwise specified, the term “rituximab biosimilar” refers to monoclonal antibody drugs developed by other companies that imitate rituximab. These biosimilar drugs have amino acid sequences identical to rituximab and exhibit similar physicochemical properties, therapeutic effects, pharmacokinetics, and safety profiles as the original innovator drug rituximab.

### Small-Molecule Toxin

Small molecule toxins suitable for use in the present invention are compounds with high cytotoxicity. Specifically, small molecule toxin is one or more monomethylauristatin (MMA); better yet small molecule toxin is monomethylauristatin-E (MMAE), monomethylauristatin-D (MMAD), monomethylauristatin-F (MMAF), or a combination thereof.

Wherein, the molecular structure of MMAE is shown below:

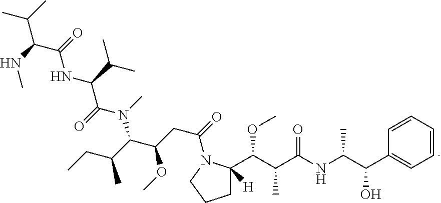 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC)C(C)C)C(C)C]

### Linker

A linker (L) suitable for use in the present invention is used to connect an antibody of the present invention to a small molecule toxin. Specifically, Linker is Maleimidocaproyl-valine-citrulline-p-aminobenzoyloxycarbonyl. e.g., as shown in the formula below:

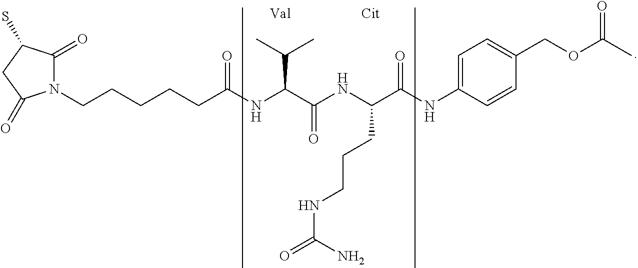 [SMILES: CC(=O)OCc1ccc(NC(=O)[C@H](CCCNC(N)=O)NC(=O)[C@@H](NC(=O)CCCCCN2C(=O)C[C@H](S)C2=O)C(C)C)cc1]

### Antibody-Drug Conjugates

The present invention provides an Antibody-drug conjugate comprising (a) a recombinant anti-CD20 monoclonal antibody linked by a linker (L) to (b) a small molecule toxin having cytotoxic properties.

As used herein, the terms “Antibody-drug conjugate of the invention” or “ADC of the invention” are used interchangeably, means a coupled drug that combines an antibody of the invention and a small molecule toxin connected through a linker.

Specifically, the antibody-drug conjugate structure is shown in formula I:

mAb−(L−D)n I

wherein,

mAb denotes an antibody of the present invention;

D denotes a small molecule toxin of the present invention;

L is the linker that connects the antibody to the small molecule toxin;

n is the average number of small molecule toxins coupled to the antibody and n is an integer or non-integer of 4.2±1; and

“-” is a chemical bond.

In another preferred example, n is an integer or non-integer of 4.2±0.5.

In another preferred example, n is an integer or non-integer of 4.2±0.3.

In another preferred example, the structure of the Antibody-drug conjugate is shown in the following formula:

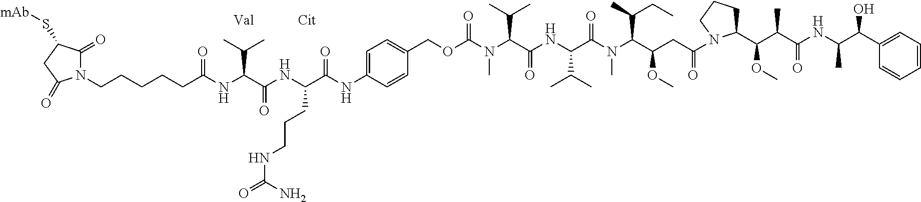

Preparation method:

The present invention provides a method of coupling an antibody and a drug such as a small molecule toxin to form an antibody-drug conjugate, which couples a small molecule toxin to an antibody through a specific connector and is capable of substantially increasing the killing capacity of the antibody against tumor cells without changing the affinity of the antibody.

First, the small molecule toxin is connected to the linker by conventional chemical synthesis, and commercially available small molecule toxins connected to the linker can be used.

Then, free sulfhydryl groups formed by reduction of four pairs of interchain disulfide bonds of a recombinantly expressed monoclonal antibody molecule is connected with the maleimide group in a linker to which a small molecule toxin has been connected. The resulting monoclonal antibody-drug conjugates have a mixture of multiple drug-loading numbers in solution due to heterogeneity of reaction sites. The possible toxin binding sites and drug loading numbers of the monoclonal antibody-drug conjugates formed are shown in FIG. 5.

As can be seen from FIG. 5, there are five theoretically formed forms of monoclonal antibody-drug conjugates carrying different drug quantities, with 0, 2, 4, 6 and 8 drug molecules. In order to assess their drug carrying quantity, an internationally recognized assessment method can be used, i.e., counting their average toxin drug to antibody molar ratio (Drug Antibody Ratio, DAR).

The antibody-drug conjugate of the present invention has a DAR value for an integer or non-integer of 4.2±1; preferably, an integer or non-integer of 4.2±0.5, more preferably, 4.2±0.3.

The present invention also provides a method of treating a disease in a mammal using an antibody-drug conjugate of the present invention. Preferably, disease is a disease that provides cells expressing CD20, such as a tumor, e.g., a lymphoma (e.g., B-cell non-Hodgkin's lymphoma) or a leukemia (chronic lymphocytic leukemia).

The present invention also provides a pharmaceutical composition (e.g., an antitumor drug) comprising an antibody-drug conjugate of the present invention.

The pharmaceutical composition comprises an effective amount of an antibody-drug conjugate according to the invention (as active ingredient), and at least one pharmaceutically acceptable carrier, diluent or excipient. When prepared, the active ingredient is usually mixed with an excipient, or diluted with an excipient, or encapsulated in a carrier that can be present in the form of a capsule or pharmaceutical capsule. When the excipient acts as a diluent, it may employ a solid, semi-solid or liquid material as the excipient, carrier or medium for the active ingredient. Thus, the composition may be a solution, a sterilized injectable solution, etc.

Suitable excipients include without limitation: lactose, glucose, sucrose, sorbitol, mannitol, starch, microcrystalline cellulose, polyvinylpyrrolidone, cellulose, water, etc.

The pharmaceutical composition may further include: wetting agents, emulsifiers, preservatives (e.g., methyl hydroxybenzoate and propyl ester), etc.

The pharmaceutical compositions may be made in unitary dosage forms or multiple dosage forms, each comprising a predetermined amount of the Antibody-drug conjugate of the present invention calculated to produce the desired therapeutic efficacy, as well as a suitable pharmacological excipient.

The pharmaceutical compositions may be administered by conventional routes, including (but not limited to): parenteral, intramuscular, intraperitoneal, intravenous, subcutaneous, intradermal, topical administration, etc.

The use of the pharmaceutical composition is to administer a safe and effective amount of antibody-drug conjugate to a subject in need thereof, such as a human being, wherein the safe and effective amount preferably ranges from 0.001 to 3 mg/kg body weight, and more preferably from 0.01 to 2 mg/kg body weight. Of course, the exact dosage should also take into account the route of administration, the health condition of the patient and other factors which are within the skill of the skilled practitioner.

In addition, the antibody-drug conjugates of the present invention may be used in combination with other therapeutic agents, including (but not limited to): cyclophosphamide, doxorubicin, vincristine, prednisone, PD1 antibodies, CTLA4 inhibitors, or combinations thereof.

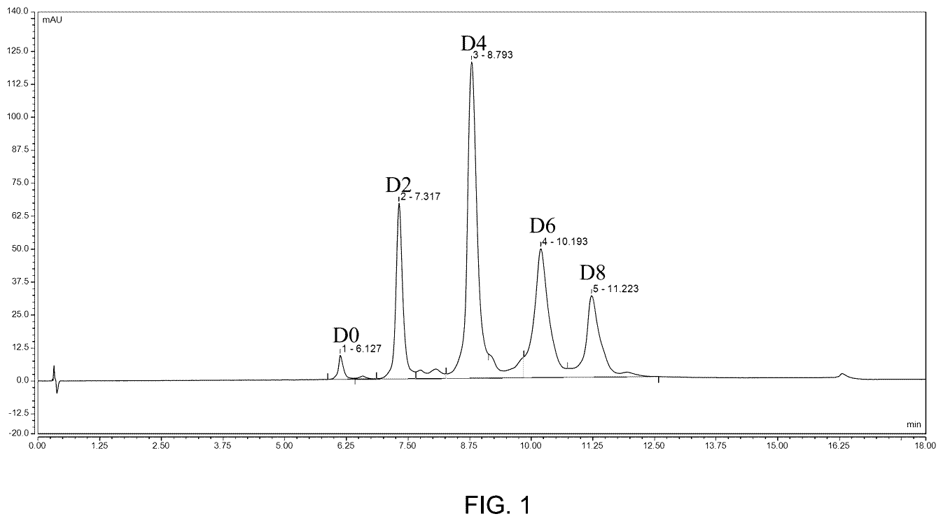

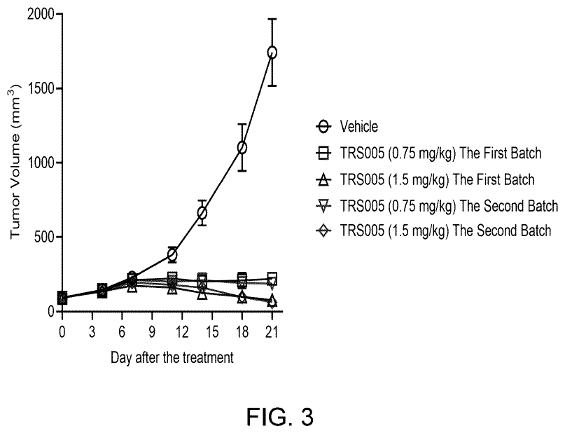

DAR value: the person skilled in the art will be aware that an ADC drug is a mixture of products having different DAR values and that the DAR value of an ADC is an average value; as shown in FIG. 1 of the present application, the ADC of the present invention has a high percentage of DAR4 component in addition to the characteristic average DAR value of 4.2. This is different from those disclosed in literature “Efficient elimination of CD103-expressing cells by anti-CD103 antibody drug conjugates in immunocompetent mice” by Yuanyi Mang et al. which describes a M290-MC-vc-PAB-MMAE ADC with a DAR value of 3.97, as can be seen in FIG. 3 of this literature, although the DAR value is 3.97, the DAR 4 component has a low percentage and is dominated by the percentage of DAR0 and DAR6 components.

As disclosed in Neelie Zacharias et al, “A homogeneous high-DAR antibody-drug conjugate platform combining THIOMAB antibodies and XTEN polypeptides”, Sparse sampling of TXCs (and TDCs) over the course of the xenograft studies indicated that the high-DAR TXCs did not have qualitatively different pharmacokinetics compared to TDC controls. Enhanced in vivo antibacterial efficacy of DAR18 TXC One day after i.v. infection with S. aureus USA300, mice were treated with a single i.v. dose of aWTA/XTEN144-dmDNA3118 (DAR18TXC, blue), aWTA/dmDNA 312 (DAR2 TDC, black) or free mAb (purple). Four days after infection, the bacterial load in the kidneys was determined by CFU assay. (A) When treated with an equimolar dose of either 23 nmoles of mAb per kg (i.e., nmoles of conjugate per kg) or 210 nmoles of mAb per kg, DAR18 TXC showed a significantly greater reduction in renal CFU compared to DAR2 TDC. In the present studies, it was able to use a dose of an antibacterial TXC that was approximately 9-fold lower than that of a low DAR ADC while maintaining comparable efficacy, thus bridging the potency gap between antibiotic and cytotoxic payloads.

It can be seen from above, the effects of different coupling, DAR values and their component distributions of the conjugates on drug efficacy is unpredictable.

### The Beneficial Effects of the Present Invention Compared with Prior Arts

The present invention provides an antibody-drug conjugate with a specific DAR component distribution optimized for drug loading, which has excellent drug activity, good stability, low toxicity, and good drug-developing properties.

It is generally accepted by those of ordinary skill in the art that antibody-drug conjugates with equal DAR values have similar efficacy, but the present invention was surprised to discover superior tumor cell killing effects with superior efficacy of antibody-drug conjugates with the specific DAR component distribution of the present invention.

It is generally believed by those of ordinary skill in the art that antibody-drug conjugates with high DAR values have more significant and excellent efficacy compared to antibody-drug conjugates with low DAR values, but the present invention surprisingly found that the antibody-drug conjugates of the present invention with a specific DAR component distribution have comparable inhibitory effects on tumor cells compared to the high DAR-valued samples, and that the use of antibody-drug conjugates with low DAR values of the present invention has comparable efficacy to that of high DAR while also reducing the risk of toxin safety and saving process costs, which has better clinical effectiveness and safety potential. The use of the low DAR antibody-drug conjugate of the present invention, while having comparable efficacy to the high DAR, also reduces the risk of toxin safety, saves process and manufacturing costs, and has better clinical effectiveness and safety potential.

The present invention provides an antibody-drug conjugate based on sulfhydryl group conjugating technology and a pharmaceutical product thereof, which produces an antibody-drug conjugate with a specific DAR component distribution accurately controlled by controlling the feed ratio; the percentage range of the distribution of each DAR component for each DAR value of the antibody-coupled product is calculated from the peak area of the hydrophobic interaction chromatography (HIC): 1.66-5.50% for DAR 0, 0.23-1.32% for DAR 1, 15.78-26.11% for DAR 2, 1.88-5.19% for DAR 3, 35.54-43.04% for DAR 4, 18.87-25.36% for DAR 6, and 9.08-14.84% for DAR 8; and the mean DAR values were 3.98-4.66. The proportion of naked antibody in the product produced by this method was less than 3%, and the effective advantage of this antibody drug with specific DAR component distribution was also demonstrated by in vitro and in vivo efficacy tests.

### Example 1 Preparation of Antibody (TRS001)

Coding sequences are fully synthesized based on the amino acid and nucleotide sequences of light and heavy chains of rituximab. The suspension-adapted CHO-K1 (from ATCC, USA) were used as the host cells. Monoclonal antibody light chain (LC) and heavy chain (HC) dual expression plasmids, i.e. pcDNA 3-RX-Neo and pcDNA 3-RX-GS, were constructed using pcDNA 3.0-based expression vector. Both expression plasmids carry genes for the light and heavy chains of the antibody (the light and heavy chains of TRS001 are completely identical to the light and heavy chain sequences of commercially available rituximab) and carry corresponding selection markers. The host cells were co-transfected with the two expression plasmids and stable transfected cells were selected with double selection markers. And then several candidate monoclonal cell lines were screened step by step using semi-solid medium cloning method. Monoclonal cell lines used for production of antibody (TRS001) were then selected by shaking flask and reactor cultivation.

The monoclonal cell line was sequenced, and sequencing results showed that the sequences encoding the light and heavy chains of TRS001 were completely identical to those of commercially available rituximab. Moreover, the physical and chemical characteristics such as molecular weight of produced rituximab biosimilar are the same as those of rituximab.

The monoclonal cell line selected by the above steps was used as the final production cell line, and a recombinant anti-human CD20 monoclonal antibody TRS001 was prepared by referring to the production method provided by rituximab original medicine patent (CN93121424.6) for subsequent examples.

### Example 2 Preparation of Antibody-Drug Conjugate (TRS005, DAR is Approximately 4.2)

### 1. Preparation of Solutions and Materials

Buffer A: 0.05 mol/L PB reaction buffer (pH 7.6)

A 0.2 mol/L PB storage buffer (pH 7.6) was prepared using a solution of 0.2 mol/L NaH2PO4·H2O and 0.2 mol/L Na2HPO4·7H2O (using Millipore purified water), filtrated through a 0.22 μm membrane (Nalgene Rapid-Flow Unit) and stored at 4° C., and RT equilibrated and diluted to 0.05 mol/L as a reduction buffer.

Reducing agent: 100 mmol/L TCEP solution

1.43 g of TECP (tris(2-carboxyethyl)phosphine) was dissolved in 50 mL of pure water to form a 100 mmol/L solution, which was equally divided to 1 ml/bottle and stored at −80° C.

Buffer B: 10 mg/mL L-cysteine termination buffer

1 g of L-cysteine was dissolved in 100 ml of a 0.1 mM DTPA (diethylenetriaminepentaacetic acid) solution, and the obtained solution was filtered through a 0.22 μm membrane and stored at −80° C.

50% ACN solution

A 50% acetonitrile solution was prepared by mixing the same volume of ACN and ultrapure water, and filtered through a 0.22 −m membrane and stored at 4° C.

Buffer C: HT preparation buffer

1 L HT preparation buffer contains 3.18 g of L-histidine, 70 g of trehalose dihydrate, and 0.2 mL of Tween 80. The pH was adjusted to 6.5 using 0.5 mol/L hydrochloric acid, and the obtained buffer was filtered through a 0.22 μm membrane and stored at 4° C.

### 2. Reduction of TRS001 (Molar Ratio: mAb:TCEP≈1:3)

The TRS001 stock solution (55 mg/mL) prepared in Example 1 was thawed overnight at 25° C. (stabilization chamber). 1616 mL of buffer A was added in a reactor (the reactor was pre-filled with 0.1 mol/L NaOH for more than 24 hours and cleaned), then 2 mL of 100 mmol/L TCEP solution was added and mixed (100 rpm, 5 minutes). 182 mL of TRS001 stock solution was added and stirred at 100 rpm, and the reduction reaction continued at 25° C. for 90 min.

3. Binding (Molar Ratio: mAb:vcMMAE≈1:7.5)

The temperature-controlled reaction bath was set to 4° C. (added with ice for cooling). The stirring speed was kept at 100-200 rpm. 13.2 mL of vcMMAE (50 mg/mL) (purchased from Levena Biopharma company) was added to 186.8 mL of 50% ACN, and then the solution was carefully added through the sample addition tube and mixed very slowly (the sample addition time was controlled within 10 min). A II the materials were gently dropped into the reaction system obtained from the above steps. The reaction time was maintained for 1 h after the vCMMAE solution was completely fed.

### 4. Termination of the Reaction

10 mL of buffer B was added into the reactor within 10 minutes to stop the conjugating reaction. After the conjugating reaction was stopped, the next step of buffer replacement was performed.

### 5. Buffer Replacement

The conjugating solution was transferred to a TFF system (pre-soaked with 0.1 mol/L NaOH and cleaned). Buffer A was changed to buffer C and then filtrated with a 0.22 microliter Rapid-Flow device. The residual rate of buffer A in the final solution should be less than 0.25%. The TRS005 stock solution after buffer replacement can be stored at 2-8° C.

6. Based on High Performance Liquid Chromatography Testing Method Recorded in Chinese Pharmacopoeia 0512, quantitative analyses were performed using reverse chromatography, in which a surface nonpolar carrier was used as the stationary phase and a solvent more polar than the stationary phase was used as the mobile phase, distribution of drug loading with respect to the DAR values is analyzed.

The final antibody-drug conjugate TRS005 with a DAR of about 4.2 was obtained.

Thirteen batches of the antibody-drug conjugate were prepared repeatedly and tested to show a very stable distribution of drug loading for the different DAR-value components of each batch. The distribution ranges of different loadings and average DAR values were developed based on the maximum and minimum values of these batch results, which are shown in Table 1 below, and representative distribution profile is shown in FIG. 1.

TABLE 1

| | | | | | | | | Average |
|---|---|---|---|---|---|---|---|---|
| Sample name | D₀ (%) | D₁ (%) | D₂ (%) | D₃ (%) | D₄ (%) | D₆ (%) | D₈ (%) | DAR |
| Example 2 | 1.66- | 0.23- | 15.782- | 1.88- | 35.54- | 18.87- | 9.08- | 3.98- |
| | 5.50 | 1.32 | 26.11 | 5.19 | 43.04 | 25.36 | 14.84 | 4.66 |

### Example 3 Preparation of Antibody-Drug Conjugate (TRS005, DAR is Approximately 6.9)

### 1. Preparation of Solutions and Materials

buffer A: 0.05 mol/l pb reaction buffer (pH 7.6)

A 0.2 mol/L PB stock buffer (pH 7.6) was prepared using a solution of 0.2 mol/L NaH2PO4·H2O and 0.2 mol/L Na2HPO4·7H2O (using Millipore purified water), filtrated through a 0.22 μm membrane (Nalgene Rapid-Flow Unit) and stored at 4° C., and RT equilibrated and diluted to 0.05 mol/L as a reduction buffer.

Reducing agent: 100 mmol/L TCEP solution

1.43 g of TECP (tris(2-carboxyethyl)phosphine) was dissolved in 50 mL of pure water to form a 100 mmol/L solution, which was equally divided to 1 ml/bottle and stored at −80° C.

Buffer B: 10 mg/mL L-cysteine termination buffer.

1 g of L-cysteine was dissolved in 100 ml of a 0.1 mM DTPA (diethylenetriaminepentaacetic acid) solution, and the obtained solution was filtered through a 0.22 μm membrane and stored at −80° C.

50% ACN solution

A 50% acetonitrile solution was prepared by mixing the same volume of ACN and ultrapure water, and filtered through a 0.22 μm membrane and stored at 4° C.

Buffer C: HT preparation buffer

1 L HT preparation buffer contains 3.18 g of L-histidine, 70 g of trehalose dihydrate, and 0.2 mL of Tween 80. The pH was adjusted to 6.5 using 0.5 mol/L hydrochloric acid, and the obtained buffer was filtered through a 0.22 μm membrane and stored at 4° C.

### 2. Reduction of TRS001 (Molar Ratio: mAb:TCEP≈1:4.6)

The TRS001 stock solution (55 mg/mL) prepared in Example 1 was thawed overnight at 25° C. (stabilization chamber). 1616 mL of buffer A was added in a reactor (the reactor was pre-filled with 0.1 mol/L NaOH for more than 24 hours and cleaned), then 3.0 mL of 100 mmol/L TCEP solution was added and mixed (100 rpm, 5 minutes). 182 mL of TRS001 stock solution was added and stirred at 100 rpm, and the reduction reaction continued at 25° C. for 90 min.

3. Binding (Molar Ratio: mAb:vcMMAE≈1:9)

The temperature-controlled reaction bath was set to 4° C. (added with ice for cooling). The stirring speed was kept at 100-200 rpm. 15.8 mL of vcMMAE (50 mg/mL) (purchased from Levena Biopharma company) was added to 184.2 mL of 50% ACN, and then the solution was carefully added through the sample addition tube and mixed very slowly (the sample addition time was controlled within 10 min). All the materials were gently dropped into the reaction system obtained from the above steps. The reaction time was maintained for 1 h after the vcMMAE solution was completely fed.

### 4. Termination of the Reaction

10 mL of buffer B was added into the reactor within 10 minutes to stop the conjugating reaction. After the conjugating reaction was stopped, the next step of buffer replacement was performed.

### 5. Buffer Replacement

The conjugating solution was transferred to a TFF system (pre-soaked with 0.1 mol/L NaOH and cleaned). Buffer A was changed to buffer C and then filtrated with a 0.22 microliter Rapid-Flow device. The residual rate of buffer A in the final solution should be less than 0.25%. The TRS005 stock solution after buffer replacement can be stored at 2-8° C.

### 6. Quantitative Analysis Performed as Described in Section 6 of Example 2.

The final Antibody-drug conjugate TRS005 with a DAR of about 6.9 was obtained, and the load distribution of the resultant antibody-drug conjugate is shown in Table 2 below.

TABLE 2

| | | | | | | | | [symbol] |
|---|---|---|---|---|---|---|---|---|
| Sample name | D₀ (%) | D₁ (%) | D₂ (%) | D₃ (%) | D₄ (%) | D₆ (%) | D₈ (%) | DAR |
| Example 3 | 0.90 | 0.55 | 1.98 | 1.12 | 9.04 | 22.51 | 63.90 | 6.90 |

### Example 4 Preparation of Antibody-Drug Conjugate (TRS005, DAR is Approximately 3.2)

### 1. Preparation of Solutions and Materials

Buffer A: 0.05 mol/L PB reaction buffer (pH 7.6)

A 0.2 mol/L PB storage buffer (pH 7.6) was prepared using a solution of 0.2 mol/L NaH2PO4·H2O and 0.2 mol/L Na2HPO4·7H2O (using Millipore purified water), filtrated through a 0.22 μm membrane (Nalgene Rapid-Flow Unit) and stored at 4° C., and RT equilibrated and diluted to 0.05 mol/L as a reduction buffer.

Reducing agent: 100 mmol/L TCEP solution

1.43 g of TECP (tris(2-carboxyethyl)phosphine) was dissolved in 50 mL of pure water to form a 100 mmol/L solution, which was equally divided to 1 ml/bottle and stored at −80° C.

Buffer B: 10 mg/mL L-cysteine termination buffer

1 g of L-cysteine was dissolved in 100 ml of a 0.1 mM DTPA (diethylenetriaminepentaacetic acid) solution, and the obtained solution was filtered through a 0.22 μm membrane and stored at −80° C.

50% ACN solution

A 50% acetonitrile solution was prepared by mixing the same volume of ACN and ultrapure water, and filtered through a 0.22 μm membrane and stored at 4° C.

Buffer C: HT preparation buffer

1 L HT preparation buffer contains 3.18 g of L-histidine, 70 g of trehalose dihydrate, and 0.2 mL of Tween 80. The pH was adjusted to 6.5 using 0.5 mol/L hydrochloric acid, and the obtained buffer was filtered through a 0.22 μm membrane and stored at 4° C.

### 2. Reduction of TRS001 (Molar Ratio: mAb:TCEP≈1:2.8)

The TRS001 stock solution (55 mg/mL) prepared in Example 1 was thawed overnight at 25° C. (stabilization chamber). 1616 mL of buffer A was added in a reactor (the reactor was pre-filled with 0.1 mol/L NaOH for more than 24 hours and cleaned), then 1.8 mL of 100 mmol/L TCEP solution was added and mixed (100 rpm, 5 minutes). 182 mL of TRS001 stock solution was added and stirred at 100 rpm, and the reduction reaction continued at 25° C. for 90 min.

3. Binding (Molar Ratio: mAb:vcMMAE≈1:6)

The temperature-controlled reaction bath was set to 4° C. (added with ice for cooling). The stirring speed was kept at 100-200 rpm.10.5 mL of vcMMAE (50 mg/mL) (purchased from Levena Biopharma company) was added to 189.5 mL of 50% A CN, and then the solution was carefully added through the sample addition tube and mixed very slowly (the sample addition time was controlled within 10 min). All the materials were gently dropped into the reaction system obtained from the above steps. The reaction time was maintained for 1 h after the vCM MAE solution was completely fed.

### 4. Termination of the Reaction

10 mL of buffer B was added into the reactor within 10 minutes to stop the conjugating reaction. After the conjugating reaction was stopped, the next step of buffer replacement was performed.

### 5. Buffer Replacement

The conjugating solution was transferred to a TFF system (pre-soaked with 0.1 mol/L NaOH and cleaned). Buffer A was changed to buffer C and then filtrated with a 0.22 microliter Rapid-Flow device. The residual rate of buffer A in the final solution should be less than 0.25%. The TRS005 stock solution after buffer replacement can be stored at 2-8° C.

The final antibody-drug conjugate TRS005 with a DAR of about 3.2 was obtained.

### Example 5 Preparation of Antibody-Drug Conjugate (TRS005, DAR is Approximately 4.2) (Compared with Example 2, the DAR Value is the Same, but the Drug Loading Distribution is Different)

### 1. Preparation of Solutions and Materials

Buffer A: 0.05 mol/L PB reaction buffer (pH 7.6)

A 0.2 mol/L PB stock buffer (pH 7.6) was prepared using a solution of 0.2 mol/L NaH2PO4·H2O and 0.2 mol/L Na2HPO4·7H2O (using Millipore purified water), filtrated through a 0.22 μm membrane (Nalgene Rapid-Flow Unit) and stored at 4° C., and RT equilibrated and diluted to 0.05 mol/L as a reduction buffer.

Reducing agent: 100 mmol/L TCEP solution

1.43 g of TECP (tris(2-carboxyethyl)phosphine) was dissolved in 50 mL of pure water to form a 100 mmol/L solution, which was equally divided to 1 ml/bottle and stored at −80° C.

Buffer B: 10 mg/mL L-cysteine termination buffer

1 g of L-cysteine was dissolved in 100 ml of a 0.1 mM DTPA (diethylenetriaminepentaacetic acid) solution, and the obtained solution was filtered through a 0.22 μm membrane and stored at −80° C.

50% ACN solution

A 50% acetonitrile solution was prepared by mixing the same volume of A CN and ultrapure water, and filtered through a 0.22 μm membrane and stored at 4° C.

Buffer C:

HT preparation buffer

1 L HT preparation buffer contains 3.18 g of L-histidine, 70 g of trehalose dihydrate, and 0.2 mL of Tween 80. The pH was adjusted to 6.5 using 0.5 mol/L hydrochloric acid, and the obtained buffer was filtered through a 0.22 μm membrane and stored at 4° C.

### 2. Reduction of TRS001 (Molar Ratio: mAb:TCEP≈1:2.5)

The TRS001 stock solution (55 mg/mL) prepared in Example 1 was thawed overnight at 25° C. (stabilization chamber). 485 mL of buffer A was added in a reactor (the reactor was pre-filled with 0.1 mol/L NaOH for more than 24 hours and cleaned), then 1.6 mL of 100 mmol/L

TCEP solution was added and mixed (100 rpm, 5 minutes). 182 mL of TRS001 stock solution was added and stirred at 100 rpm, and the reduction reaction continued at 25° C. for 90 min.

3. Binding (Molar Ratio: mAb:vcMMAE≈1:5.6)

The temperature-controlled reaction bath was set to 4° C. (added with ice for cooling). The stirring speed was kept at 100-200 rpm. 9.8 mL of vcMMAE (50 mg/mL) (purchased from Levena Biopharma company) was added to 186.8 mL of 50% ACN, and then the solution was carefully added through the sample addition tube and mixed very slowly (the sample addition time was controlled within 10 min). All the materials were gently dropped into the reaction system obtained from the above steps. The reaction time was maintained for 1 h after the vcMMAE solution was completely fed.

### 4. Termination of the Reaction

10 mL of buffer B was added into the reactor within 10 minutes to stop the conjugating reaction. After the conjugating reaction was stopped, the next step of buffer replacement was performed.

### 5. Buffer Replacement

The conjugating solution was transferred to a TFF system (pre-soaked with 0.1 mol/L NaOH and cleaned). Buffer A was changed to buffer C and then filtrated with a 0.22 microliter Rapid-Flow device. The residual rate of buffer A in the final solution should be less than 0.25%. The TRS005 stock solution after buffer replacement can be stored at 2-8° C.

### 6. Quantitative Analysis Performed as Described in Section 6 of Example 2.

The final Antibody-drug conjugate TRS005 with a DAR of about 4.2 was obtained, and the load distribution (DAR component distribution) of the antibody-drug conjugate is shown in Table 3 below.

TABLE 3

| Sample | | | | | | | | Average |
|---|---|---|---|---|---|---|---|---|
| name | D₀ (%) | D₁ (%) | D₂ (%) | D₃ (%) | D₄ (%) | D₆ (%) | D₈ (%) | DAR |
| Example 5 | 11.83 | 0.97 | 13.71 | 2.76 | 22.14 | 45.62 | 2.97 | 4.23 |

Example 2: DAR Value 4.2, DAR Component Distribution Within the Preferred Distribution of the invention; the molar ratio of mAb: TCEP is: 1:3; the feed ratio of mAb: vcM MA E is: 1:7.5, and the feed ratios of the Example 2 are within the following preferred feed ratios: the molar ratio of mAb: TCEP is from 1:2.9 to 1:3.1; the molar ratio of recombinant anti-CD20 monoclonal antibody to vcM MAE is in the range of from 1:7.0 to 1:8.0, and it is possible to obtain the distribution of the specific DAR components of the antibody-drug conjugates;
Example 3: DAR value of 6.9, the DAR value is not within the preferred range of the present invention; the molar ratio of mAb:TCEP is: 1:4.6; the feed ratio of mAb:vcMMAE is: 1:9, and the feed ratios of the Example 3 are not within the following preferred feed ratios: the molar ratio of mAb:TCEP is 1:2.9 to 1:3.1; the molar ratio of recombinant anti-CD20monoclonal antibody to vcMMAE in the range of 1:7.0 to 1:8.0;
Example 4: DAR value of 3.2, the DAR value is not within the preferred range of the present invention; the molar ratio of mAb:TCEP is: 1:2.8; the feed ratio of mAb:vcMMAE is: 1:6, and the feed ratios of the Example 4 are not within the following preferred feed ratios: the molar ratio of mAb:TCEP of 1:2.9 to 1:3.1; the molar ratio of recombinant anti-CD20monoclonal antibody to vcMMAE is not in the range of 1:7.0 to 1:8.0;
Example 5: DAR value of 4.2, the distribution of DAR components is not within the preferred distribution of the present invention; the feed ratio of mAb:TCEP is: 1:2.5; the molar ratio of mAb:vcMMAE is: 1:5.6, and the feed ratios of the Example 5 are not within the following preferred feed ratios: the molar ratio of mAb:TCEP is in the range of 1:2.9 to 1:3.1; the molar ratio of recombinant anti-CD20 monoclonal antibody vs. vcMMAE is not in the range of a molar ratio of 1:7.0 to 1:8.0;

The present invention provides a method for preparing antibody conjugate based on sulfhydryl coupling and producing specific DAR component distributions by controlling specific feed ratios. The proportion of uncoupled small molecules in the product produced by this method was less than 3%, and the effectiveness of the antibody-drug conjugate with specific DAR component distribution was also demonstrated by in vivo and in vitro efficacy tests.
Example 6 Lyophilized preparation of recombinant anti-CD20 monoclonal antibody-drug conjugate

The preparation comprises:

Rituximab, histidine hydrochloride, alginic acid, mannitol, polysorbate 80, and water for injection.

The preparations obtained were tested by stability experiments and the results indicate that they fully meet the requirements of pharmaceutical preparations.

### Experiment 1: Method for Internalization Efficacy of Antibody-Drug Conjugate (TRS005) by Flow Cytometry

### 1. Reagents and Cells

Sheath fluid: filtered deionized water;

DPBS (1x): purchased from gibco, product number: 14190-136;

Anti-human IgG antibody (FITC), purchased from abcam, product number: ab81051;

Raji cells were purchased from the cell bank of the Typical Culture Preservation Committee of the Chinese Academy of Sciences, catalog number: TCHu44.

Ramos cells were purchased from COBIOER BIOSCIENCES CO., LTD.

### 2. Experimental Equipment

BD Accuri C6 flow cytometer: purchased from BD Biosciences.

### 3. Method

Instrument start-up: Place a tube of freshly purified water at the inlet needle of the flow cytometer, turn on the instrument, rinse the instrument with water in Fast mode for 5 minutes, prepare 8-peaks and 6-peaks quality control samples according to the instrument manual, and calibrate the four channels of the flow cytometer, and then standby.

Cell preparation: take logarithmic growth phase Raji cells or Ramos cells, centrifuge at 1000 rpm for 5 minutes, resuspend using DPBS, adjust the density of live cells to 2×106 cells/mL, and divide into 1.5 mL centrifuge tubes, 1 mL/tube.

Sample dilution: The sample to be tested (TRS005 with a DAR of approximately 4.2) was diluted to 1000 μg/mL, 10 μL was added to each 1 mL of cell suspension, and blown and mixed to make the final concentration of the sample 10 μg/mL.

Incubation the cell suspension to which the samples were added was placed on ice and ice-bath for 30 minutes.

Endocytosis: at the end of the ice bath, the cell suspension was centrifuged at 1500 rpm for 5 min, the supernatant was discarded, resuspended using 1 mL of pre-cooled DPBS, and dispensed in a volume of 200 uL/tube into four 1.5 mL centrifuge tubes, one as a negative control group placed on ice in an ice bath, and three as a parallel experimental group placed in a water bath at 37° C. Incubate for 2 h.

Staining: the secondary anti-human IgG antibody (FITC labeled) was diluted 100-fold using DPBS. At the end of incubation, the cell suspension was centrifuged at 1500 rpm for 5 minutes, the supernatant was discarded and resuspended using 250 μL of pre-cooled DPBS, 50 μL of the diluted secondary antibody solution was added to each of the suspensions, blown to mix, and placed on ice in a freezer bath for 30 minutes.

Instrumental Testing at the end of the ice bath, the cell suspension was centrifuged at 1500 rpm for 5 minutes, the supernatant was discarded, and resuspended using 200 μL of pre-cooled DPBS and assayed on the machine.

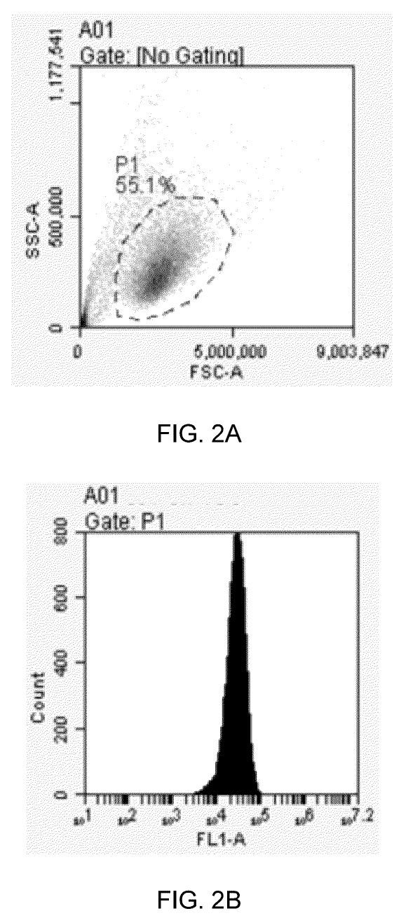

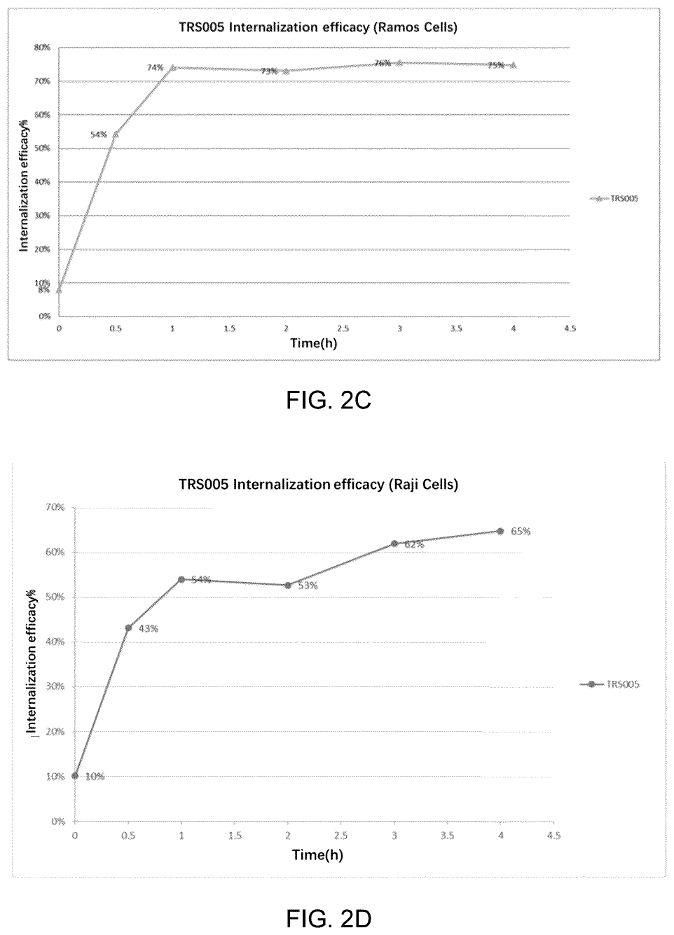

Cells from the negative control group treated with ice bath were gating and the average fluorescence intensity of cells in each tube at FL1 was recorded. Flow cytometry scatter plots and single-parameter histograms of the cells are shown in FIG. 2A and FIG. 2B, respectively.

The percentage of endocytosis of the samples was calculated based on the average fluorescence intensity according to the following formula.

Internalization efficacy ratio %=(mean fluorescence intensity of negative control group−mean fluorescence intensity of experimental group)/mean fluorescence intensity of negative control group×100%.

The internalization efficacy results of Ramos cells and Raji cells are shown in FIG. 2C and FIG. 2D, respectively. The results showed that the Antibody-drug conjugate TRS005 of the present invention had a good internalization efficacy.

### Experiment 2: In vitro Cytotoxicity Assay

### 1. Reagents

RPM 11640, purchased from gibco, product number 11875-085;

FBS, purchased from Lonsera, product number S711-001S;

Streptomycin/Penicillin Dual Antibody, purchased from HyClone, Lot: SV30010;

DPBS (Dulbecco's Phosphate Buffer Solution) was purchased from Gibco, product number 14190-136;

CCK-8 reagent, purchased from Biolite, product number 35004.

### 2. Cells

Ramos cells were purchased from COBIOER BIOSCIENCES CO., LTD.

### 3. Method

Cell culture and plating: Ramos cells with viability greater than 80% in the logarithmic growth phase up to 30 generations were selected for the experiment. Ramos cells were removed from culture flasks and added to 50 mL centrifuge tubes and blown to mix. 1000 rpm (≈188g) centrifugation was performed for 5-6 minutes. The supernatant was discarded and the cells were resuspended with the appropriate volume of diluent. For counting, adjust the cell density with pre-warmed complete medium (RPM11640 +10% FBS+1% double antibody (streptomycin & penicillin)) to approximately 2×105 cells/mL for cell spreading, add 50 μL of cells to each target well, and 100 μL of sterile water or DPBS to the bordering wells. place in a CO2 incubator and incubate at 37° C. 5% CO2.

Sample dilution: The TRS005 reference (a batch of TRS005 product with a calibrated DAR of approximately 4.2) at a concentration of 80% of the dilution starting point was diluted to 6 μg/mL and 4.8 μg/mL as the samples to be tested and the QC samples, respectively, and gradient dilutions were performed in 96-well plates for 12 concentration points.

Sample addition incubation: 50 μL per well of diluted standards, samples and QC from the dilution plate were added to the cell plate. Incubate the cell plate in a CO2 incubator for about 72 hours.

Chromogenic reaction After incubation, add 10 μL of CCK-8 reagent to each well for color development, and incubate in CO2 incubator for 3-4 hours.

Plate reading: Load the plate into the enzyme labeling instrument and read the plate with 450 nm as the reading wavelength and 650 nm as the reference wavelength.

Data processing: The IC50 value was calculated using a four-parameter fitting model with the concentration value as the horizontal coordinate and the difference between the absorbance value at 450 nm minus the absorbance value at 650 nm as the vertical coordinate.

The data are shown in table 4 below:

TABLE 4

| Ramos cells | | IC₅₀ (ng/ml) |
|---|---|---|
| Example 2 (DAR of | Parallel detection 1 | 58.9 |
| approximately 4.2) | Parallel detection 2 | 64.8 |
| Example 5 (DAR of | Parallel detection 1 | 133 |
| approximately 4.2, | Parallel detection 2 | 142 |
| distribution varies) | | |
| Example 3 sample (DAR | Parallel detection 1 | 55.1 |
| of approximately 6.9) | Parallel detection 2 | 52.4 |

The results in Table 4 indicate that the samples from Example 2 (with a DAR value of approximately 4.2) and Example 3 (with a DAR value of approximately 6.9) have the same killing effect on Ramos cells. It is generally believed by those of ordinary skill in the art that antibody-drug conjugates (ADCs) with a low DAR value have significantly superior efficacy compared to those with a high DAR value. However, the present invention surprisingly finds that the ADCs with a specific DAR-value drug payload distribution in the present invention have a comparable tumor cell inhibitory effect to the samples with a high DAR value. Using the ADCs with a low DAR value in the present invention not only achieves efficacy comparable to that of high-DAR ADCs but also reduces the safety risk associated with toxins and saves the process manufacturing cost, showing better potential for clinical efficacy and safety.

The average DAR values of both Example 5 and Example 2 were approximately equal to 4.2, but the DAR distributions were different. The results in Table 1 indicate that the killing effect of the Example 5 sample on Ramos cells was inferior to that of the Example 2 sample. It is generally accepted by those of ordinary skill in the art that antibody-drug conjugate with equal DAR values have similar efficacy, but the present invention was pleasantly surprised to find superior tumor cell killing effects with the specific DAR component distribution of the present invention, which has excellent efficacy.

### Experiment 3 In vivo Anti-Tumor Activity Test

1. Test animals and housing environment

Species: BALB/c nude mice

Gender of animals: male mice.

Age at acquisition: 6-8 weeks; Body weight at the beginning of the experiment: 18˜20 g.

Housing environment: SPF grade.

### 2. Cell

Daudi cells were purchased from the cell bank of the Type Culture Collection of the Chinese Academy of Sciences.

PRM 11640 medium was purchased from Corning, production batch No.04216004.

Matrigel® Matrix gel was purchased from Corning, production batch No. 2174002.

### 3. TEST SAMPLE: The Test Sample was Selected From Two Batches of the Product Prepared in Example 2.

### 4. Test Methods

Under aseptic conditions of 5% CO2 and 37° C., Daudi cells were cultured with PRMI1640 medium containing 10% fetal bovine serum, respectively, and expanded to 5.0×108 cells. Cells were collected by centrifugation and mixed 1:1 with Matrix gel and inoculated subcutaneously into the right side of the rib cage of BALB/c nude mice at 1×107 cells/200 μl/animal.

When the mean tumor volume reached 100 mm3, the group was randomized:

Negative control group: vector (Vehicle);

The first batch TRS005 (0.75 mg/kg) group: TRS005 (0.75 mg/kg);

The first batch TRS005 (1.5 mg/kg) group: TRS005 (1.5 mg/kg);

The second batch TRS005 (0.75 mg/kg) group: TRS005 (0.75 mg/kg);

The second batch TRS005 (1.5 mg/kg) group: TRS005 (1.5 mg/kg);

There were 10 nude mice in each group. Then the administration of the drug was started by tail vein administration, and the body weight of BALB/c nude mice and the long and short diameters of the tumors were measured, and recorded during the administration.

Tumor volume calculation formula:=long diameter×short diameter×short diameter/2.

### 4. Statistical Analysis

Tumor volume and Tumor weight were expressed as Mean±Standard Error of the Mean (Mean±SEM). Statistical tests were analyzed by one-way analysis of variance (ANOVA), with p<0.05 considered significant and p<0.01 considered highly significant.

### 5. Results

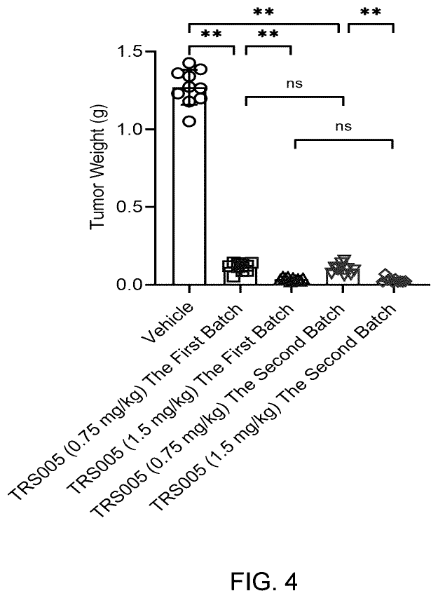

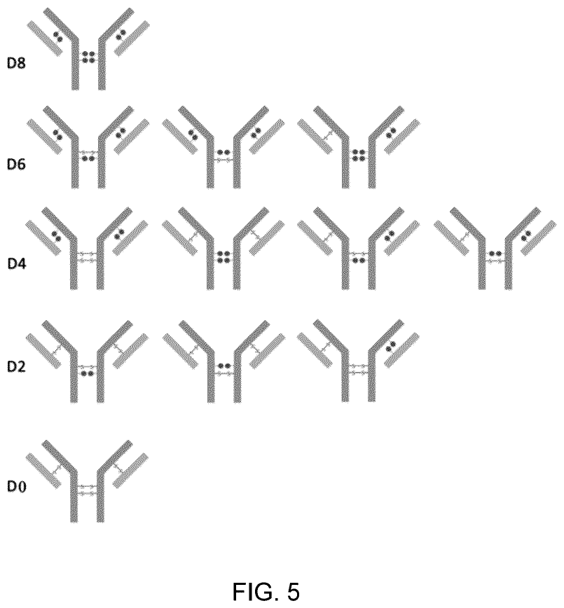

TRS005 showed a very significant therapeutic effect on the Daudi cell xenografts tumor in BALB/c nude mice model (see FIGS. 3 and 4). 91.08% and 92.09% tumor inhibition rate were observed in the TRS005 (0.75 mg/kg) group, 97.15% and 97.71% tumor inhibition rate were observed in the TRS005 (1.5 mg/kg) group, respectively.

There was no significant difference in the anti-tumor effect of the two batches of TRS005 at the same dose.

All the documents cited herein are incorporated into the invention as reference, as if each of them is individually incorporated. Further, it would be appreciated that, in light of the above described teaching of the invention, the skilled in the art could make various changes or modifications to the invention, and these equivalents would still be in the scope of the invention defined by the appended claims of the application.

What is claimed is:

1. An antibody-drug conjugate with a structure as shown in formula I or pharmaceutically acceptable salt thereof:
mAbμ(LμD)n I
wherein,
mAb represents an anti-CD20 antibody;
D represents a small molecule toxin;
L is a linker connecting the anti-CD20 antibody and the small molecule toxin, wherein the linker is selected from maleimidocaproyl-valine-citrulline-p-aminobenzyloxycarbonyl;
Antibody-drug conjugate consists of a mixture of conjugates with diverse DAR values;
n is an average number of the small molecule toxins conjugated to the antibody;
“-” is a bond
wherein an average ratio of small molecule toxin to antibody (DAR) of the antibody-drug conjugate is in a range of 4.2±0.5.

2. The antibody-drug conjugate or a pharmaceutically acceptable salt thereof of claim 1, wherein the antibody-drug conjugate comprises a mixture of components with different DAR values:
DAR0, DAR1, DAR2, DAR3, DAR4, DAR6, DAR8, and wherein main DAR components in the antibody-drug conjugate has a percentage range of: 14-28% for component of DAR 2, 34-44% for component of DAR 4, 17-26% for component of DAR 6, and 9-15% for component of DAR 8, based on DAR values calculated from peak areas of hydrophobic interaction chromatography (HIC) for the antibody-drug conjugate.

3. The antibody-drug conjugate or a pharmaceutically acceptable salt thereof of claim 1, wherein the anti-CD20 antibody is rituximab or a biosimilar thereof.

4. The antibody-drug conjugate or a pharmaceutically acceptable salt thereof of claim 1, wherein average DAR value is 3.98-4.66.

5. The antibody-drug conjugate or a pharmaceutically acceptable salt thereof of claim 1, wherein the antibody-drug conjugate has a structure shown as the following formula: 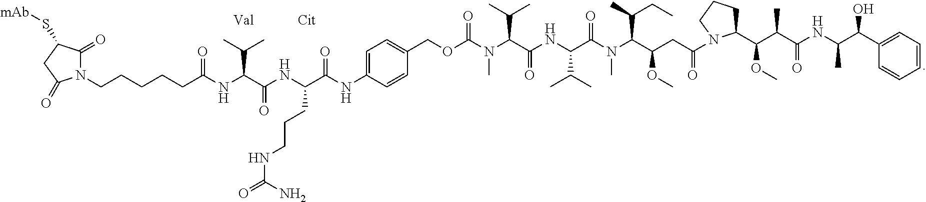
wherein mAb is rituximab or a biosimilar thereof, and/or wherein the average DAR value is 3.98-4.66.

6. The antibody-drug conjugate or a pharmaceutically acceptable salt thereof of claim 1, wherein the DAR components in the antibody-drug conjugate has a percentage range of: 1.66-5.50% for component of DAR 0, 0.23-1.32% for component of DAR 1, 15.78-26.11% for component of DAR 2, 1.88-5.19% for component of DAR 3, 35.54-43.04% for component of DAR 4, 18.87-25.36% for component of DAR 6, and 9.08-14.84% for component of DAR 8, based on DAR values calculated from the peak areas of hydrophobic interaction chromatography (HIC) for the antibody-drug conjugate.

7. The antibody-drug conjugate or a pharmaceutically acceptable salt thereof of claim 1, wherein the small molecule toxin is one or more monomethylauristatin selected from monomethylauristatin-E (MMAE), monomethylauristatin-D (MMAD), monomethylauristatin-F (MMAF), or a combination thereof.

8. The antibody-drug conjugate or a pharmaceutically acceptable salt thereof of claim 1, wherein the small molecule toxin is monomethyl auristatin-E (MMAE).

9. The antibody-drug conjugate or a pharmaceutically acceptable salt thereof of claim 8, the specified component of the conjugate with 4 MMAE had the highest percentage in the conjugate mixture and the proportion of naked antibodies not conjugated with MMAE was less than 3%.

10. The antibody-drug conjugate or a pharmaceutically acceptable salt thereof of claim 8, wherein component of DAR 4 comprises 35-43% of the antibody-drug conjugate, and wherein a naked antibody without conjugation with MMAE comprises less than 3% of the antibody-drug conjugate.

11. A method for the preparation of an antibody-drug conjugate targeting CD20 or a pharmaceutically acceptable salt thereof, comprising steps of:
(1) reducing a recombinant anti-CD20 monoclonal antibody and a reducing agent in a PB reaction buffer, the reducing agent was tris(2-carboxyethyl)phosphine (TCEP), and a molar ratio of recombinant anti-CD20 monoclonal antibody to reducing agent was 1:2.9 to 1:3.1;
(2) adding a mixture of vcMMAE and solvent dropwise to the reaction system of step (1) so that the reduced recombinant anti-CD20 monoclonal antibody and vcMMAE undergo a conjugation reaction; wherein the molar ratio of recombinant anti-CD20 monoclonal antibody to vcMMAE was 1:7.0 to 1:8.0;
(3) terminating the conjugation reaction by adding termination buffer to the reaction system of step (2); and
(4) after termination of the conjugation reaction in step (3), performing buffer exchange, transferring the conjugate solution to a TFF system, changing the PB reaction buffer to preparation buffer and then filtering.

12. The method of claim 11, wherein the molar ratio of recombinant anti-CD20 monoclonal antibody to tris(2-carboxyethyl)phosphine (TCEP) is 1:3; and the molar ratio of recombinant anti-CD20 monoclonal antibody to vcMMAE is 1:7.5.

13. The method of claim 11, wherein in step (1), the recombinant anti-CD20 monoclonal antibody and the reducing agent are subjected to a reduction reaction in PB reaction buffer at pH 7.6 for 90 min at 25° C., and the molar ratio of recombinant anti-CD20 monoclonal antibody to tris(2-carboxyethyl)phosphine (TCEP) is 1:3; and wherein in step (2), the reduced recombinant anti-CD20 monoclonal antibody and vcM MAE are subjected to a conjugation reaction at 4° C. for 60 min, and the molar ratio of recombinant anti-CD20 monoclonal antibody to vcM MAE is 1:7.5.

14. The method of claim 11, wherein the method comprises steps of:
(1) subjecting the recombinant anti-CD20 monoclonal antibody and the reducing agent to a reduction reaction in a PB reaction buffer at pH 7.6 for 90±10 minutes at 25±1° C. to obtain a reaction system containing the reduced recombinant anti-CD20 monoclonal antibody; wherein the recombinant anti-CD20 monoclonal antibody is rituximab or its biosimilars, and wherein the reducing agent is tris(2-carboxyethyl)phosphine, and the molar ratio of the recombinant anti-CD20 monoclonal antibody to the reducing agent is 1:2.9 to 1:3.1;
(2) adding a solution of vcM MA E in acetonitrile and water dropwise to the reaction system of step (1) so that the reduced recombinant anti-CD20 monoclonal antibody and vcM MAE is subjected to a conjugation reaction for 60±10 minutes at 4±0.5° C.; the volume ratio of acetonitrile to water was 1:1, and the molar ratio of recombinant anti-CD20 monoclonal antibody to vcMMAE is from 1:7.0 to 1:8.0;
(3) adding a L-cysteine termination buffer to the reaction system of step (2) to terminate the conjugation reaction, wherein the L-cysteine termination buffer is prepared by dissolving L-cysteine in DTPA solution; and
(4) after terminating the conjugation reaction in step (3), buffer exchange was performed by transferring the conjugate solution to the TFF system, changing the PB reaction buffer to preparation buffer, and then filtering.

15. The method of claim 11, wherein the formulation buffer was HT formulation buffer, and HT formulation buffer 1 L contained L-histidine 3.18 g, alginate dihydrate 70 g, and Tween 80 0.2 mL, pH was adjusted to 6.5 using 0.5 mol/L hydrochloric acid, and filtered through 0.22 μm membrane.

16. A pharmaceutical composition comprising the antibody-drug conjugate or a pharmaceutically acceptable salt thereof of claim 1, and a pharmaceutically acceptable carrier, a diluent, a stabilizer or an excipient.

17. The pharmaceutical composition of claim 16, wherein the pharmaceutically acceptable carrier comprises water, a buffer, histidine hydrochloride, alginate and tween 80.

18. A method for treating a tumor in a subject in need thereof, comprising administrating the antibody-drug conjugate or a pharmaceutically acceptable salt thereof of claim 1 or the pharmaceutical composition of claim 16 to the subject.

19. The method of claim 18, wherein the tumor comprises a CD20 positive tumor or CD20-expressing tumor, lymphoma or leukemia.

20. The method of claim 18, wherein the tumor comprises B-cell lymphoma, non-Hodgkin's lymphoma or chronic lymphocytic leukemia.
